(19) United States
(12) Patent Application Publication
(10) Pub. No.: US 2007/0088052 A1
(43) Pub. Date: Apr. 19, 2007

(54) Sulfonamide derivatives as 5ht7 receptor antagonists

(75) Inventors: Eric Lattmann, Birmingham (GB); Isidro Merino Ferdandez, Navarra (ES)

Correspondence Address: FISH & RICHARDSON P.C., PO BOX 1022, MINNEAPOLIS, MN, 55440-1022, US

(73) Assignee: Aston University, Birmingham (GB)

(21) Appl. No.: 10/564,689

(22) PCT Filed: Jun. 29, 2004
(86) PCT No.: PCT/GB04/02788

§ 371 Date: Sep. 13, 2006

(30) Foreign Application Priority Data
Jul. 10, 2003 (GB) 0316128.8

(51) Int. Cl. A61K 31/445; C07D 211/06

(52) U.S. Cl. 514/317; 546/229; 514/319; 546/206

(57) ABSTRACT

The present invention discloses a compound of formula (I):—wherein A is an aromatic moiety or selected from benzyl, C₁-C₁₆ alkyl dialkylamino, dialkylaminoalkyl, alkoxyalkyl, cyano, and mono-, di-, or tri-hydroxyalkyl and/or aryl, B is an aromatic moiety, R₁ and R₂ are independently C₁ to C₆ alkyl or NR₁R₂ forms a 5 to 8 membered ring optionally containing one or two additional heteroatoms selected from nitrogen, oxygen and sulphur and which is optionally substituted by C₁ to C₆ alkyl, and n is 0 or 1, and salts and hydrates thereof. The invention is also concerned with methods of synthesising the compounds and their uses as 5-HT₇ ligands.

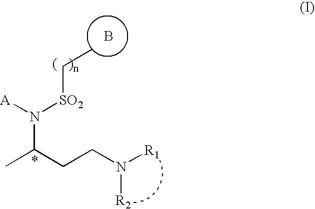

The present invention relates to novel sulfonamide compounds which have pharmacological activity, a process for their production and their use in the treatment of CNS disorders.

The 5-HT₇ receptor has been identified as a target for the development of useful therapeutic agents. In particular, 5-HT₇ receptor antagonists may be therapeutically effective in the treatment of CNS disorders such as anxiety, depression, schizophrenia and sleep disorders. WO 97/49695 and WO 00/56712 both disclose a series of sulfonamide derivatives which are claimed to have 5-HT₇ receptor antagonist activity. Forbes et. al. (J. Med. Chem. 1998, 41, 665) discloses sulphonamide compounds which are selective 5-HT₇ receptor antagonists.

It is an object of the present invention in a first aspect to provide a novel series of sulfonamide compounds which preferably have binding affinity for the 5-HT₇ receptor.

According to the present invention there is provided a compound of formula (I): 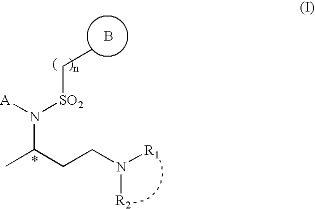
wherein A is an aromatic moiety or selected from benzyl, C₁-C₁₆ alkyl (eg. methyl, t-butyl and dodecyl), dialkylamino (eg. dimethylamino), dialkylaminoalkyl (eg. dimethylaminoethyl), alkoxyalkyl (eg. methoxy), cyano, and mono-, di-, or tri-hydroxyalkyl and/or aryl (eg. tris(hydroxyethyl)methyl, and 2-phenyl-2-hydroxyethyl),
B is an aromatic moiety,
R₁ and R₂ are independently C₁ to C₆ alkyl or NR₁R₂ forms a 5 to 8 membered ring optionally containing one or two additional heteroatoms selected from nitrogen, oxygen and sulphur and which is optionally substituted by C₁ to C₆ alkyl, and
n is 0 or 1.

The scope of the invention also extends to salts, particularly physiologically acceptable salts and hydrates of the compounds of formula (I)

Preferably, the moiety NR₁R₂ is 4-methylpiperidinyl.

Each of A (when an aromatic moiety) and B may be phenyl, naphthyl, azobenzene, or a 5 or 6 membered heteroaryl ring or a benzofused heteroaryl ring containing from 1 to 3 heteroatoms selected from oxygen, nitrogen and sulphur, any of which may be optionally substituted with one or more of C₁₋₆ alkyl (preferably C₁₋₃), C₁₋₆ alkoxy (preferably C₁₋₃), C₁₋₆ alkylthio, halo, cyano, nitro, C₁₋₆ alkylcarbonyl (preferably C₁₋₃), and trifluoromethyl.

As used herein, expressions relating to chains of carbon atoms such as “alkyl” and “alkoxy” include within their scope both straight and branched moieties.

Preferably, A is phenyl, benzyl, naphth-1-yl or pyridin-2-yl. When A is phenyl, said phenyl is preferably monosubstituted. Preferred substituents include cyano (preferably o- or p-substituted), methoxy (preferably m-substituted), acetyl (p-substituted), nitro (p-substituted) and methyl (p-substituted).

More preferably, A is p-toluidine, m-anisidine or naphth-1-yl. Most preferably, A is m-anisidine.

Preferably, B is phenyl, naphth-1-yl or thiophen-2-yl. When B is phenyl, said phenyl is preferably mono- or disubstituted. Preferred substituents include methyl (preferably m-substituted), methoxy (preferably dimethoxy), nitro (preferably m-substituted), bromo, trifluoromethyl, acetamido and phenyl (preferably p-substituted).

Most preferably B is m-toluidine, naphth-1-yl, m-nitrophenyl, 4-biphenyl or m,p-dimethoxyphenyl.

When n is 1, B is preferably phenyl. Preferably, n is 0.

Particularly preferred compounds are:—

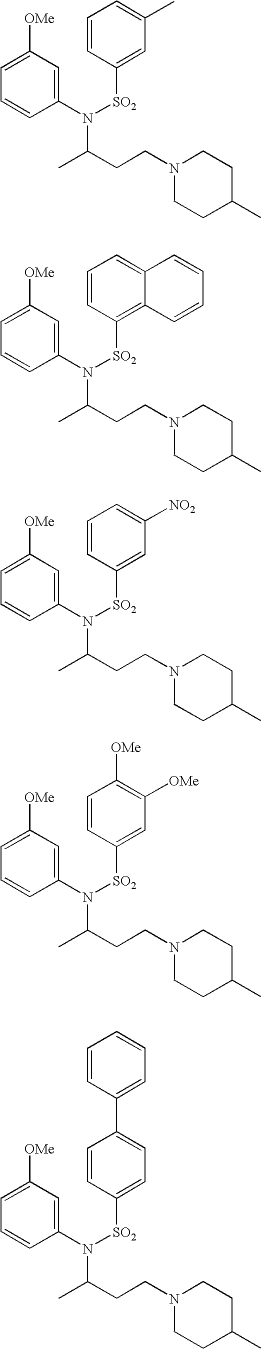

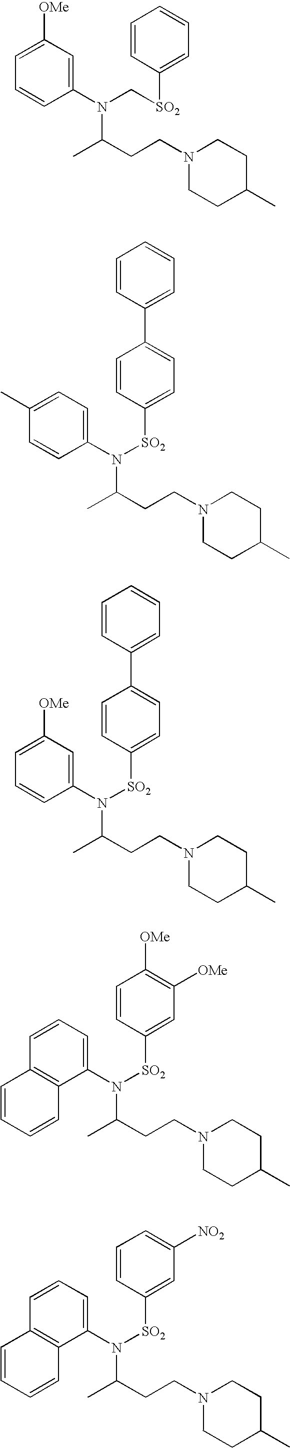

It will be understood that formula (I) is intended to embrace all possible isomers, including optical isomers and mixtures thereof, including racemates.

Specifically, the C atom marked “*” in formula (I) is a chiral centre. From previous studies it is likely that (R) stereochemistry at this centre will be preferred. In addition, the present invention includes within its scope prodrugs of the compounds of formula (I). In general, such prodrugs will be functional derivatives of the compounds of formula (I) which are readily convertible in vivo into, the required compound of formula (I). Conventional procedures for the selection and preparation of suitable prodrug derivatives are well known to the skilled person and are described, for example, in “Design of Prodrugs”, ed H. Bungaard, Elsevier, 1985.

The pharmaceutically acceptable salts of the compounds of formula (I) include the conventional non-toxic salts or the quaternary ammonium salts of the compounds of formula (I) formed, e.g., from non-toxic inorganic or organic acids. The pharmaceutically acceptable salts of formula (I) also include those formed from a base, such as an alkali or alkaline earth metal hydroxide, or an organic base, such as an amine or a quaternary ammonium hydroxide.

The present invention also resides in a method of synthesising a compound of formula (I) comprising the steps of
(i) coupling a compound of formula (II) with a compound of formula (III) or coupling a compound of formula (IV) with a compound of formula (V), 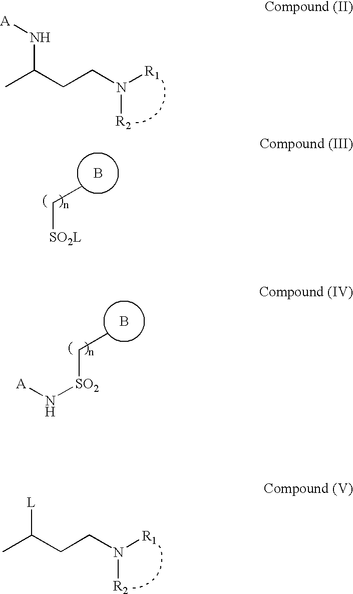
where L is a leaving group and A, B and n are as defined in formula (I),
(ii) removing any protecting groups which may be present and
(iii) optionally forming a pharmaceutically acceptable salt.

Suitable leaving groups for L include halogen (particularly chloro and iodo). For compound (III) Y is preferably chloro and for compound (V) L is preferably iodo.

During the synthesis, it may be necessary to protect certain functional groups. The use of protecting groups including methods for their attachment and cleavage are well known to the skilled organic chemist and are described in, for example T. W. Greene “Protective Groups in Organic Synthesis”, Wiley, New York (1981).

Compound (IV) is conveniently reacted as a salt, for example sodium salt.

The compounds of formulae (II) to (V) can be prepared by the methods described herein or by analogous methods to those known to the skilled person.

The present invention also resides in the use of a compound of the first aspect as a 5-HT₇ receptor ligand and/or as a 5-HT₇ receptor antagonist.

Preferably, the compounds of the present invention exhibit selectivity towards the 5-HT₇ receptor over one or more other 5-HT receptor subtypes.

The 5-HT₇ antagonist activity of the compounds of formula (I) makes these compounds useful as pharmacological agents for mammals, especially humans, for the treatment and prevention of CNS disorders and other indications.

Therefore the present invention in a third aspect resides in a method of treatment of a mammal afflicted with a CNS disorder, or prophylaxis in a mammal at risk of such a CNS disorder by administration of a therapeutically effective amount of a compound in accordance with the first aspect of the present invention.

The invention also resides in a pharmaceutical formulation comprising a compound of the first aspect of the present invention in admixture with a pharmaceutically acceptable carrier therefor.

The invention further resides in the use of a compound of the first aspect in the preparation of a medicament, particularly a medicament for the treatment or prophylaxis of a CNS disorder, or other indications including inflammation, spastic colon, renal disorders, hypotension, cardiovascular shock, stroke, septic shock and gastrointestinal conditions such as irritable bowel syndrome.

Examples of CNS disorders against which the compounds of the present invention may be effective include anxiety, depression, sleep disorders, migraine, Parkinson's disease, schizophrenia, pain and appetite disorders.

The dosage administered to a patient will normally be determined by the prescribing physician and will generally vary according to the age, weight and response of the individual patient, as well as the severity of the patient's symptoms. However, in most instances, an effective therapeutic daily dosage will be in the range of from about 0.05 mg/kg to about 50 mg/kg of body weight and, preferably, of from 0.5 mg/kg to about 20 mg/kg of body weight administered in single or divided doses. In some cases, however, it may be necessary to use dosages outside these limits.

While it is possible for an active ingredient to be administered alone as the raw chemical, it is preferable to present it as a pharmaceutical formulation. The formulations, both for veterinary and for human medical use, of the present invention comprise an active ingredient in association with a pharmaceutically acceptable carrier therefor and optionally other therapeutic ingredient(s). The carrier(s) must be ‘acceptable’ in the sense of being compatible with the other ingredients of the formulation and not deleterious to the recipient thereof.

Conveniently, unit doses of a formulation contain between 0.1 mg and 1 g of the active ingredient. Preferably, the formulation is suitable for administration from one to six, such as two to four, times per day. For topical administration, the active ingredient preferably comprises from 1% to 2% by weight of the formulation but the active ingredient may comprise as much as 10% w/w. Formulations suitable for nasal or buccal administration, such as the self-propelling powder-dispensing formulations described hereinafter, may comprise 0.1 to 20% w/w, for example about 2% w/w of active ingredient.

The formulations include those in a form suitable for oral, ophthalmic, rectal, parenteral (including subcutaneous, vaginal, intraperitoneal, intramuscular and intravenous), intra-articular, topical, nasal or buccal administration.

Formulations of the present invention suitable for oral administration may be in the form of discrete units such as capsules, cachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary or paste. For such formulations, a range of dilutions of the active ingredient in the vehicle is suitable, such as from 1% to 99%, preferably 5% to 50% and more preferably 10% to 25% dilution. Depending upon the level of dilution, the formulation will be either a liquid at room temperature (in the region of about 20° C.) or a low-melting solid.

Formulations for rectal administration may be in the form of a suppository incorporating the active ingredient and a carrier such as cocoa butter, or in the form of an enema.

Formulations suitable for parenteral administration comprise a solution, suspension or emulsion, as described above, conveniently a sterile aqueous preparation of the active ingredient that is preferably isotonic with the blood of the recipient.

Formulations suitable for intra-articular administration may be in the form of a sterile aqueous preparation of the active ingredient, which may be in a microcrystalline form, for example, in the form of an aqueous microcrystalline suspension or as a micellar dispersion or suspension. Liposomal formulations or biodegradable polymer systems may also be used to present the active ingredient particularly for both intra-articular and ophthalmic administration.

Formulations suitable for topical administration include liquid or semi-liquid preparations such as liniments, lotions or applications; oil-in-water or water-in-oil emulsions such as creams, ointments or pastes; or solutions or suspensions such as drops. For example, for ophthalmic administration, the active ingredient may be presented in the form of aqueous eye drops, as for example, a 0.1-1.0% solution.

Drops according to the present invention may comprise sterile aqueous or oily solutions. Preservatives, bactericidal and fungicidal agents suitable for inclusion in the drops are phenylmercuric salts (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the eye. An eye lotion may comprise a sterile aqueous solution optionally containing a bactericide or preservative prepared by methods similar to those for the preparation of drops. Lotions or liniments for application to the skin may also include an agent to hasten drying and to cool the skin, such as an alcohol, or a softener or moisturizer such as glycerol or an oil such as castor oil or arachis oil.

Creams, ointments or pastes according to the present invention are semi-solid formulations of the active ingredient in a base for external application. The base may comprise one or more of a hard, soft or liquid paraffin, glycerol, beeswax, a metallic soap; a mucilage; an oil such as a vegetable oil, e.g. almond, corn, arachis, castor or olive oil; wool fat or its derivatives; or a fatty acid ester of a fatty acid together with an alcohol such as propylene glycol or macrogols. The formulation may also comprise a suitable surface-active agent, such as an anionic, cationic or non-ionic surfactant such as a glycol or polyoxyethylene derivatives thereof. Suspending agents such as natural gums may be incorporated, optionally with other inorganic materials, such as silicaceous silicas, and other ingredients such as lanolin.

Formulations suitable for administration to the nose or buccal cavity include those suitable for inhalation or insufflation, and include powder, self-propelling and spray formulations such as aerosols and atomisers. The formulations, when dispersed, preferably have a particle size in the range of 10 to 200μ.

Such formulations may be in the form of a finely comminuted powder for pulmonary administration from a powder inhalation device or self-propelling powder-dispensing formulations, where the active ingredient, as a finely comminuted powder, may comprise up to 99.9% w/w of the formulation.

Self-propelling powder-dispensing formulations preferably comprise dispersed particles of solid active ingredient, and a liquid propellant having a boiling point of below 18° C. at atmospheric pressure. Generally, the propellant constitutes 50 to 99.9% w/w of the formulation whilst the active ingredient constitutes 0.1 to 20% w/w, for example, about 2% w/w, of the formulation. Formulations of the present invention may also be in the form of a self-propelling formulation wherein the active ingredient is present in solution. Such self-propelling formulations may comprise the active ingredient, propellant and co-solvent, and advantageously an antioxidant stabiliser. Suitable co-solvents are lower alkyl alcohols and mixtures thereof. The co-solvent may constitute 5 to 40% w/w of the formulation, though preferably less than 20% w/w of the formulation. Antioxidant stabilisers may be incorporated in such solution-formulations to inhibit deterioration of the active ingredient and are conveniently alkali metal ascorbates or bisulphites. They are preferably present in an amount of up to 0.25% w/w of the formulation.

Formulations of the present invention may also be in the form of an aqueous or dilute alcoholic solution, optionally a sterile solution, of the active ingredient for use in a nebuliser or atomiser, wherein an accelerated air stream is used to produce a fine mist consisting of small droplets of the solution. Such formulations usually contain a flavouring agent such as saccharin sodium and a volatile oil. A buffering agent such as sodium metabisulphite and a surface-active agent may also be included in such a formulation which should also contain a preservative such as methylhydroxybenzoate.

Other formulations suitable for nasal administration include a powder, having a particle size of 20 to 500 microns, which is administered in the manner in which snuff is taken, i.e. by rapid inhalation through the nasal passage from a container of the powder held close up to the nose.

In addition to the aforementioned ingredients, the formulations of this invention may include one or more additional ingredients such as diluents, buffers, flavouring agents, binders, surface active agents, thickeners, lubricants, preservatives e.g. methylhydroxybenzoate (including anti-oxidants), emulsifying agents and the like. A particularly preferred carrier or diluent for use in the formulations of this invention is a lower alkyl ester of a C₁₈ to C₂₄ mono-unsaturated fatty acid, such as oleic acid, for example ethyl oleate. Other suitable carriers or diluents include capric or caprylic esters or triglycerides, or mixtures thereof, such as those caprylic/capric triglycerides sold under the trade name Miglyol, e.g. Miglyol 810.

The invention will now be further described by way of example only.
Preparation of Compounds of Formula (II) and (III) and Coupling Thereof (Scheme (I))

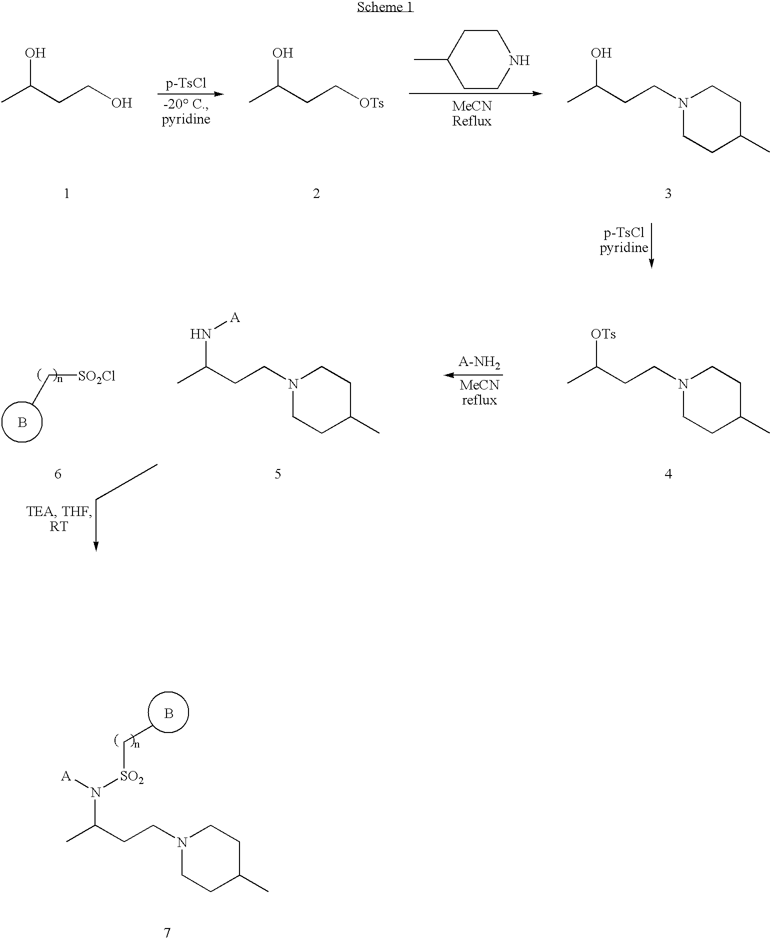

The diol 1 was mono-tosylated selectively (Hintzer, K.; Koppenhoefer, B.; Schurig, V; J. Org. Chem., 1982, 47, 3850-3854) with p-toluene sulfonylchloride in pyridine at −20° C. giving the 3-hydroxybutyl tosylate 2 in excellent purity (Tercio, J.; Ferreira, B.; Simonelli, F; Tetrahedron 1990, 46, 6311-6318). Tosylate 2 was refluxed in acetonitrile with methylpiperidine providing the amino-alcohol building block 3 in good yield. For purification the amino-alcohols may be recrystallized and the by-products can be washed out easily using an automated liquid handling system. Activation of the second hydroxyl group of diol 1 was carried out by tosylation of aminoalcohol 3 with p-TsCl in pyridine at room temperature to give tosylate 4 which may be used in situ for the subsequent reaction with primary amines to synthesize the propylendiamines 5. The sulphonamides 7 were synthesized by reacting propylendiamines 5 with sulfonylchlorides 6 and TEA in THF at room temperature.
Preparation of Compounds of Formula (IV) and (V) and Coupling Thereof (Scheme (II)) 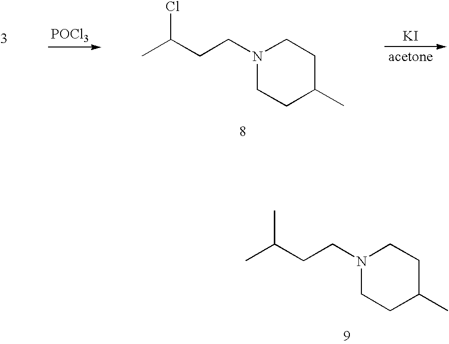
Amine 1-24 [Table 1]

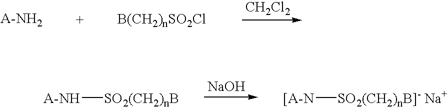

Sulphonyl Chloride A-O [Table 2]

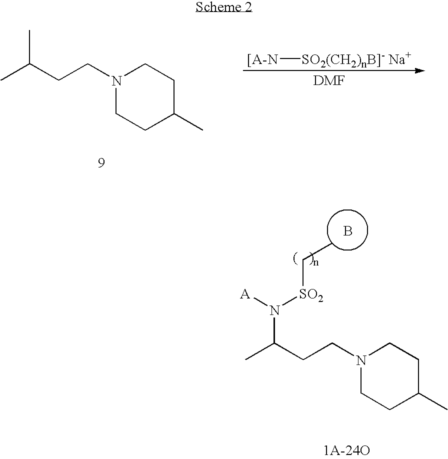

In this convergent synthesis the sulphonamide was prepared and alkylated using the chemically reactive iodide 9 prepared from alcohol 3 (scheme 1). The alcohol 3 was converted into alkyl chloride 8 using phosphorus oxychloride (POCl₃) at 0° C. with overnight stirring at room temperature. The alkyl chloride 8 did not react with the sulfonamides in good yields so was converted to the iodide 9 with potassium iodide in refluxing acetone.

The chemical and physical properties of the alkyl iodide 9 were very important for the development of an automated synthesis, since it is a solid at room temperature and can be purified easily by recrystallization in dichloromethane/petrolether. In addition, the compound is not soluble in ethyl ether, whereas most of the final compounds (sulphonamides A1-O24) were reasonably soluble in this solvent. Thus, an automated technique was performed to extract the compounds in ether without extracting the starting material.

TABLE 1
Overview of amines applied to the combinatorial synthesis

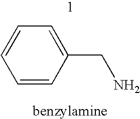

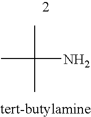

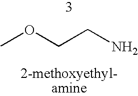

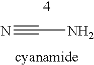

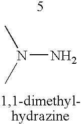

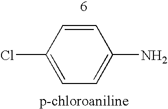

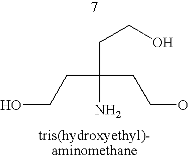

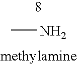

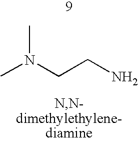

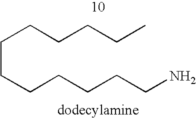

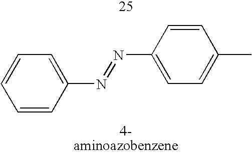

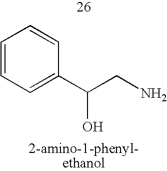

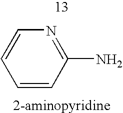

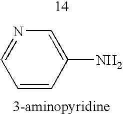

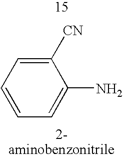

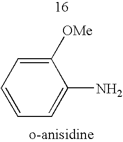

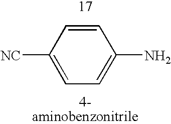

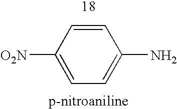

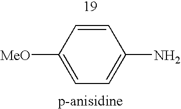

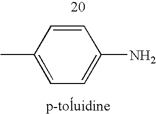

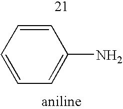

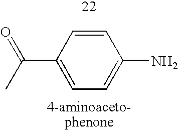

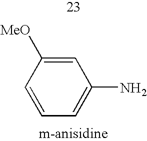

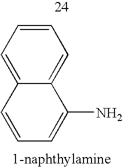

The sulfonyl building block [sulphonamide] was synthesized by reacting an aromatic sulfonyl chloride A-O (Table 2) with a primary amine 1-24 (Table 1) in dichloromethane and triethylamine as catalyst to afford the sulfonamide intermediates, which were purified by washing with Na₂CO₃. The sulfonamides reacted with a solution of sodium hydroxide yielding their sodium salts, which were soluble in water, and therefore could be easily purified. The alkyl iodide 9 reacted with the sodium salt of the sulfonamide intermediates in DMF at 100° C. to afford a 2-dimensional combinatorial library of the desired sulfonamides A1-O24.

TABLE 2
Selected Sulfonyl chlorides (A-O)

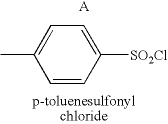

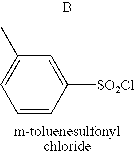

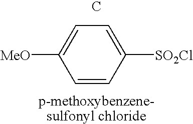

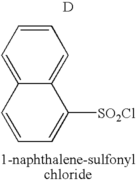

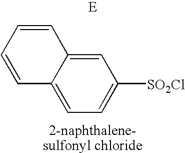

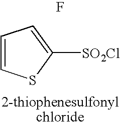

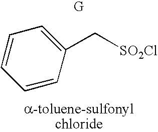

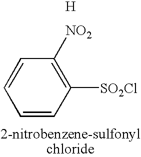

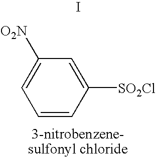

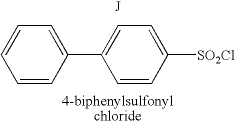

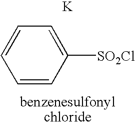

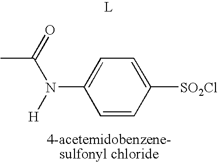

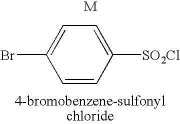

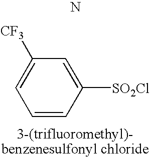

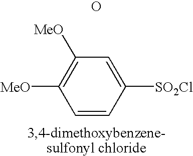

Material and Methods

Atmospheric pressure chemical ionisation mass spectrometry (APCI-MS) was carried out on a Hewlett-Pachkard 5989B quadrupole instrument connected to an electrospray 59987A unit with an APCI accessory and automatic injection using Hewlett-Packard 1100 series autosampler. IR spectra were recorded as KBr disc on a Mattson 3000 FT-IR spectrophotometer. Proton NMR spectra were obtained on a Bruker AC 250 instrument operating at 250 MHz, with TMS as internal standard.

Automated Synthesis of Combinatorial Libraries (Scheme 2)

The sulfonyl chlorides were dissolved in dichloromethane (1 mmol in 3 ml of CH₂Cl₂) or in 1,4-dioxane when the sulfonyl chloride is not soluble in dichloromethane. The amines were also dissolved in dichloromethane (1 mmol in 3 ml of CH₂Cl₂) and 1.5 mmol of triethylamine per mmol of amine was added to the solution. The solutions of the amines and the sulfonyl chlorides were mixed in vials placed on a rack by an automated pippeting system. The compounds were allowed to react for 2 days. They were washed first with water (3 ml. 2 times, to extract the triethylamine hydrochloride) and later with Na₂CO₃ (1 N. 3 ml. 2 times) to extract the unreacted sulfonyl chloride. A solution of NaOH (0.5 N 2 ml) was used to extract the sulfonamides as sodium salts. The samples (water phase) were subsequently dried. A solution of alkyl iodide 9 in DMF was added to every sample of sulfonamide sodium salt by the automated pippeting system. The samples were heated at 100° C. for 30 h. All the DMF was lost in the process and a yellow to brown oil remained in the vials. The samples were washed with a solution of NaOH (1 N. 3 ml. 3 times) to remove the excess of starting sulfonamide. Then, the compounds were extracted with ethyl ether (5 ml. several times) and dried. As the alkyl iodide 9 is not soluble in ether, only the final compounds were extracted.

### EXAMPLE 1

### N-Benzyl-N-[1-methyl-3-(4-methyl-piperidin-1-yl)-propyl]-benzenesulfonamide

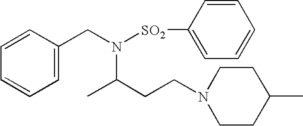

MS m/z (%): 415 (M+1, 100).

IR (film, cm⁻¹): νₘₐₓ 3061, 3028, 2926, 2867, 1638, 1445, 1336, 1148, 868, 723, 689. ¹H-NMR (CDCl₃, δ, ppm): 0.88-0.98 (m, 6H), 1.06 (dd, 3H, J₁=6.8 Hz, J₂=3.9 Hz, CH₃CNSO₂), 1.50-1.61 (m, 3H), 2.03-2.16 (m, 1H), 2.35-2.51 (m, 5H), 2.71-2.81 (m, 1H), 3.46 (s, 2H, CH₂Ph), 4.20-4.44 (m, 3H), 4.69 (dd, 1H, J₁=15.5 Hz, J₂=4.9 Hz), 7.23-7.42 (m, 8H,Ph+C₄H+C₅H+C₆H), 7.63 (s, 1H, C₂H).

¹³C-NMR (CDCl₃, δ): 18.4, 21.5, 30.9, 33.6, 34.4, 40.3, 41.9, 45.6, 48.6, 51.7, 124.0, 127.3, 127.5, 128.2, 128.3, 128.4, 128.9, 133.1, 138.0, 139.1, 140.9, 168.1

Receptor Binding Assay, ^{[3]}H-5-CT

The combinatorial library was screened using radiolabeled 5-carboxamidotryptamine, a selective 5-HT-7 agonist. Tissues were homogenized in ice cold sucrose (0.32 M, 25 ml) for 15 strokes at 500 rpm and centrifuged at 13000 rpm for 10 mins. The supernatant was re-centrifuged at 13000 rpm for 20 mins. The resulting pellet was re-dispersed to the required volume of buffer at 500 rpm and stored in aliquots at 70° C.

Binding was achieved using radioligand 5-carboxamidotryptamine at 25 pM. The samples were incubated {with membranes (0.1 mg/ml)} in 20 mM Hepes, 1 mM EGTA, 5 mM MgCl₂, 150 mM NaCl, at pH 6.5 for 2 hrs at RT and then centrifuged at 11000 rpm for 5 minutes. The membrane pellets were washed twice with water and the bound radioactivity was measured in a Packard Cobra Auto-gamma counter (B5005). All binding assays were carried out with SB-258719 as standard. Controls (no compound) were also added. All samples were made in duplicate and repeated twice. All compounds were initially screened for percentage inhibition at 10 μM. Samples showing an average inhibition of <35% were diluted to 1 μM and re-screened and if active diluted again. The results are outlined in Table 3.

TABLE 3
5HT-7 receptor binding data using ^{[3]}H-5-CT

| | | | | | | | | | | | | |
|---|---|---|---|---|---|---|---|---|---|---|---|---|
| A1 | B1 | C1 | D1 * | E1 * | F1 | G1 | H1 | I1 ** | J1 | M1 | N1 | O1 |
| A3 | B3 | C3 | D3 | E3 * | F3 | G3 | H3 | I3 | J3 | M3 | N3 ** | O3 |
| A6 | B6 | C6 ** | D6 | E6 | F6 | G6 | H6 | I6 | J6 | M6 | N6 | O6 |
| A8 | B8 | C8 | D8 | E8 | F8 | G8 | H8 | I8 ** | J8 | M8 | N8 | IO8 |
| A9 | B9 * | C9 | D9 | E9 | F9 | G9 | H9 | FI9 | J9 | M9 * | N9 | O9 |
| A10 | B10 | C10 | D10 | E10 | F10 | G10 * | H10 | I10 | J10 | M10 | N10 | O10 |
| A13 | B13 | C13 | D13 ** | E13 | F13 | G13 | H13 | I13 | J13 | M13 | N13 | O13 |
| A16 | B16 ** | C16 | D16 | E16 | F16 | G16 | H16 | I16 | J16 | M16 | N16 ** | O16 |
| A20 | B20 | C20 | D20 | E20 | F20 | G20 | H20 | I20 | J20 *** | M20 | N20 ** | O20 |
| A21 | B21 | C21 | D21 | E21 | F21 | G21 | H21 | I21 ** | J21 | M21 | N21 | O21 |
| A22 | B22 | C22 | D22 | E22 | F22 | G22 * | H22 | I22 | J22 | M22 | N22 | O22 |
| A23 | B23 *** | C23 | D23 *** | E23 | F23 | G23 *** | H23 | I23 ** | J23 *** | M23 | N23 | O23 |
| A24 | B24 | C24 * | D24 | E24 | F24 | G24 * | H24 | I24 *** | J24 | M24 | N24 | O24 *** |

* 1 micromolar
** 100 nM
*** 10 nM dropout defined as < 35 binding affinity.

From the above, it can be seen that the m-anisidine and naphthyl-sulfonamides displayed the best binding affinity. For example, IC₅₀ values of D23, J20, J23 and O24 were calculated as 34, 61, 39, 83 nM, respectively.

Animal Studies in Mice (In Vivo Evaluation as Antidepressants)

Despair Swim Test (Immobility Time Test)

It has been suggested that rodents forced to swim in a restricted space, from which they cannot escape are induced to the characteristic behaviour of immobility. This reflects a state of despair, which can be reduced by several agents, which are therapeutically effective in human depression. Antidepressant drugs have the effect of reducing the duration of immobility.

Mice were brought into the laboratory at least one day before the experiment and were housed in separate cages, with free access to food and water. The mice were individually forced to swim inside a vertical Plexiglas cylinder (height: 40 cm; diameter: 18 cm, containing 15 cm of water maintained at 30° C.). After 10 min in the water the mice were removed and allowed to dry in a heated enclosure (32° C.) before being returned to their home cages. They were again replaced in the cylinder 24 hrs later and the total duration of the immobility was measured during a 5 min test. Floating behaviour during this 5 min period was reproducible in different groups of mice. An animal was judged to be immobile whenever it remained floating passively in the water in a slightly hunched but upright position, its nose just above the surface. The test drug or the standard were administered one hour prior to testing.

Tail Suspension Test

Male mice weighting 20-25 g were used preferentially. The were housed in plastic cages for at least 10 days prior to testing in a 12 hours light cycle with food and water freely available.

Animals were transported from the housing room to the testing area in their own cages and were allowed to adapt to the new environment for 1 hour before testing. Groups of 6 animals were treated with the test compounds or the vehicle by interperitoneal injection 30 min prior to testing. For the test the mice were suspended on the edge of a shelf 58 cm above a table top by adhesive tape, placed approximately 1 cm from the tip of the tail. The duration of immobility was recorded for a period of 5 minutes. Mice were considered immobile when they hang passively and the time was recorded.

The immobility of the swimming test was compared with the immobility in the tail suspension test, a second standard assay to test antidepressants. The results are shown in Table 4 for the selected sulfonamides J20 and O24.

| | | J20 | J20 | O24 | O24 |
|---|---|---|---|---|---|
| | 5% DMSO | 1 mg/kg | 5 mg/kg | 1 mg/kg | 5 mg/kg |
| Forced | 95 ± 12 | 42 ± 6 | 28 ± 4 | 69 ± 7 | 51 ± 11 |
| swim test | | | | | |
| (s) | | | | | |
| Tail | 144 ± 16 | 130 ± 11 | 92 ± 9 | 81 ± 8 | 74 ± 9 |
| suspension | | | | | |
| (s) | | | | | |

These selected examples were found to be active in both standard assays and the antidepressant activity is of greater magnitude than Imipramine and Fluoxetine used as standards (data not shown).

1. A compound of formula (I): 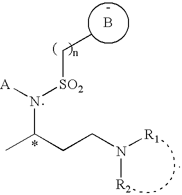
Wherein A is an aromatic moiety or selected from benzyl, C₁-C₁₆ alkyl, dialkylamino, dialkylaminoalkyl, alkoxyalkyl, cyano, and mono-, di-, or tri-hydroxyalkyl and/or aryl,
B is an aromatic moiety,
R₁ and R₂ are independently C₁ to C₆ alkyl or NR₁R₂ forms a 5 to 8 membered ring optionally containing one or two additional heteroatoms selected from nitrogen, oxygen and sulphur and which is optionally substituted by C₁ to C₆ alkyl, and
n is 0 or 1,
and salts and hydrates thereof.

2. The compound of claim 1, wherein the moiety NR₁R₂ is 4-methylpiperidinyl.

3. The compound of claim 1, wherein A is an aromatic moiety, and A and B are independently selected from phenyl, naphthyl, azobenzene, or a 5 or 6 membered heteroaryl ring or a benzofused heteroaryl ring containing from 1 to 3 heteroatoms selected from oxygen, nitrogen and sulphur, any of which may be optionally substituted with one or more of C₁₋₆ alkyl, C₁₋₆ alkoxy, C₁₋₆ alkylthio, halo, cyano, nitro, C₁₋₆ alkylcarbonyl, and trifluoromethyl.

4. The compound of claim 1, wherein A is phenyl, benzyl, naphth-1-yl or pyridin-2-yl.

5. The compound of claim 1, wherein A has one or more of the following substituents: cyano, methoxy, acetyl, nitro or methyl.

6. The compound of claim 1 wherein A is monosubstituted phenyl.

7. The compound of claim 1, wherein A is p-toluidine, m-anisidine or naphth-1-yl.

8. The compound of claim 1, wherein B is phenyl, naphth-1-yl or thiophen-2-yl.

9. The compound of claim 1, wherein B has one or more of the following substituents: methyl, methoxy, nitro, bromo, trifluoromethyl, acetamido or phenyl.

10. The compound of claim 1, wherein B is mono- or di-substituted phenyl.

11. The compound of claim 1, wherein B is m-toluidine, naphth-1-yl, m-nitrophenyl, 4-biphenyl or m,p-dimethoxyphenyl.

12. The compound of claim 1, wherein n is 1 and B is phenyl.

13. The compound of claim 1, wherein n is 0.

14. A compound as in claim 1, having one of the following formulae:

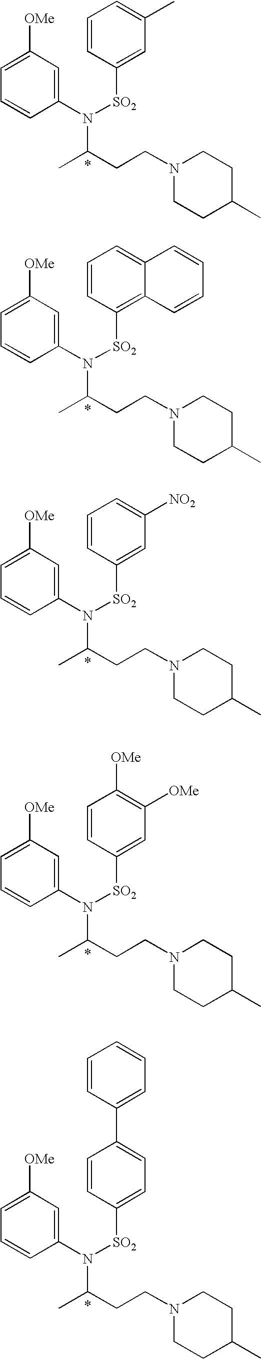

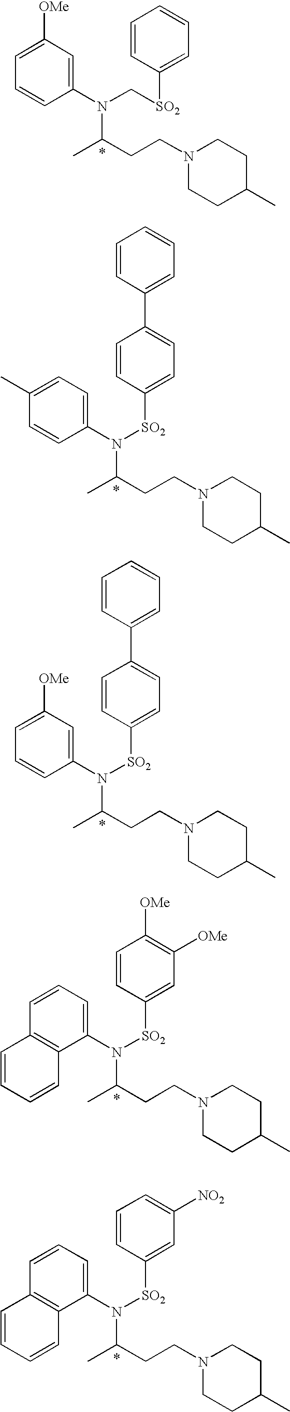

15. The compound of claim 1 which has (R) stereochemistry at C*.

16. A compound which is metabolized or otherwise converted in vivo to a compound claimed in claim 1.

17. A method of synthesizing a compound of claim 1 comprising
(i) coupling a compound of formula (II) with a compound of formula (III) or coupling a compound of formula (IV) with a compound of formula (V), 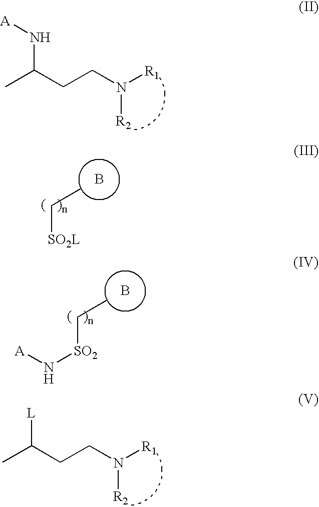
wherein L is a leaving group and A, B and n are as defined in formula (I),
(ii) removing any protecting groups which may be present and
(iii) optionally forming a pharmaceutically acceptable salt.

18. The method of claim 17, wherein L is halogen.

19. The method of claim 17, wherein compounds of formulae (II) and (III) are coupled and L is chloro.

20. The method of claim 17, wherein compounds of formula (IV) and (V) are coupled and L is iodo.

21. The use of a compound of claim 1 as a 5-HT7 receptor ligand and/or as a 5-HT7 receptor antagonist.

22. The use of claim 21, wherein said compound exhibits selectivity towards the 5-HT7 receptor over one or more other 5-HT receptor subtypes.

23. A method of treatment of a mammal afflicted with a CNS disorder, or prophylaxis in a mammal at risk of such a CNS disorder, by administration of a therapeutically effective amount of a compound of claim 1.

24. A pharmaceutical formulation comprising a compound of claim 1 in admixture with a pharmaceutically acceptable carrier therefor.

25. The use of a compound as claimed in claim 1 in the preparation of a medicament, for the treatment or prophylaxis of a CNS disorder, inflammation, spastic colon, renal disorders, hypotension, cardiovascular shock, stroke, septic shock or gastrointestinal conditions such as irritable bowel syndrome.
